(19) United States
(12) Patent Application Publication
(10) Pub. No.: US 2019/0151042 A1
(43) Pub. Date: May 23, 2019

(54) INTRAORAL POSITIONING DEVICE

(71) Applicants: Ross HOLMAN, Menlo Park, CA (US); Brian KNOTT, Palo Alto, CA (US); RADTEC MEDICAL DEVICES, INC., San Carlos, CA (US)

(72) Inventors: Ross HOLMAN, Menlo Park, CA (US); Brian KNOTT, Palo Alto, CA (US)

(21) Appl. No.: 16/097,417

(22) PCT Filed: May 3, 2017
(86) PCT No.: PCT/US17/30773

§ 371 Date: Oct. 29, 2018

Related U.S. Application Data
(60) Provisional application No. 62/331,864, filed on May 4, 2016.
(60) Provisional application No. 62/399,491, filed on Sep. 26, 2016.
(60) Provisional application No. 62/399,490, filed on Sep. 26, 2016.

(51) Int. Cl. A61B 90/16; A61N 5/10; A61B 13/00; A61C 5/00; A61C 9/00; A61C 17/06

(52) U.S. Cl. CPC A61B 90/16; A61N 5/1047; A61N 5/103; A61B 13/00; A61B 2090/101; A61C 9/0006; A61C 17/04; A61N 2005/1097; A61C 5/007

(57) ABSTRACT

An intraoral positioning device (IPD) is disclosed. The IPD is to be positioned within a patient's mouth for intensity-modulated radiation therapy (IMRT) planning and treatment. The intraoral positioning device comprises: a bite stent having upper and lower sections, the upper section configured to engage an upper oral structure of the patient's mouth and the lower structure configured to engage a lower oral structure of the patient's mouth during IMRT planning and treatment; a tongue displacement stent extending from and fixed to the bite stent, the tongue displacement stent configured to displace the tongue of the patient in a direction with respect to a base of the patient's mouth in accordance with a patient's prescription for IMRT planning and treatment, whereby the patient's tongue is positioned in a motionless state during the IMRT planning and treatment; and a deployment handle extending from and fixed to the bite stent.

### CROSS REFERENCE TO RELATIONS APPLICATIONS

This application claims priority to U.S. provisional application numbers (1) 62/331,864, filed on May 4, 2016 entitled “Intraoral Positioning Device”, (2) 62/399,491, filed Sep. 26, 2016, entitled “Intraoral Positioning Device”, (3) 62/399,490, filed on Sep. 26, 2016, entitled “Method of Making Intraoral Positioning Devices For User Radiation Therapy Treatment and Registration Device for Method,” which are all (three) incorporated by reference herein.

### FIELD OF THE INVENTION

The present invention relates to an intraoral positioning device (IPD).

### BACKGROUND OF THE INVENTION

Over the last 10 years, head and neck cancer diagnoses have increased annually by ten to fifteen percent. The rise in occurrence is largely the result of the increase in HPV infections. Intensity modulated radiation therapy treatment or IMRT is the primary treatment modality along with chemotherapy. In IMRT, patient target locations are treated with small beams of high intensity radiation that are delivered from multiple directions to conform to the shape of the tumor while avoiding healthy anatomic structures from radiation exposure. In order for IMRT to be effective, i.e., to reduce or remove malignant tumors, the patient must remain in position and motionless during many repeatable treatment sessions (e.g. 30). Prior devices for IMRT, however, have been less than effective for such purposes when it comes to positioning the lower jaw and tongue.

It would thus be advantageous to provide a device to overcome the disadvantages in such prior devices above.

### SUMMARY OF THE INVENTION

Embodiments of an intraoral positing device are disclosed.

In accordance with an embodiment of this disclosure, an intraoral positioning device to be positioned within a patient's mouth for intensity-modulated radiation therapy (IMRT) planning and treatment is disclosed, the intraoral positioning device comprising: a bite stent having upper and lower sections, the upper section configured to engage an upper oral structure of the patient's mouth and the lower structure configured to engage an lower oral structure of the patient's mouth during IMRT planning and treatment; a tongue displacement stent extending from and fixed to the bite stent, the tongue displacement stent configured to displace a tongue of the patient in a direction with respect to a base of the patient's mouth in accordance with a patient's prescription for IMRT planning and treatment, whereby the patient's tongue is positioned in a motionless state during the IMRT planning and treatment; and a deployment handle extending from and fixed to the bite stent, the handle configured to be grasped to by a user so as to introduce and deploy the intraoral device into the patient's mouth for the IMRT planning and treatment and remove the device after IMRT planning and treatment.

In accordance with another embodiment of the disclosure, an intraoral positioning device to be positioned within a patient's mouth for intensity-modulated radiation therapy (IMRT) planning and treatment is disclosed, the intraoral positioning device comprising: an upper section configured to engage an upper oral structure of the patient's mouth and a lower structure configured to engage a lower oral structure of the patient's mouth during IMRT planning and treatment; a plurality of posts spaced between the upper and lower section, thereby supporting the upper and lower sections; a tongue displacement stent configured to pivot with respect to the first and second sections so as to enable a user to adjust the position of the tongue displacement stent to a plurality of positions to displace the tongue of the patient in accordance with a patient's prescription for IMRT planning and treatment, whereby the patient's tongue is positioned in a motionless state during the IMRT planning and treatment; and a deployment handle extending from and fixed the upper or lower sections, the handle configured to be grasped to by a user so as to introduce and deploy the intraoral device into the patient's mouth for the IMRT planning and treatment and remove the device after IMRT planning and treatment.

### BRIEF DESCRIPTION OF THE DRAWINGS

FIG. 1 depicts a perspective view of an example intraoral positioning device.

FIG. 2 depicts a rear perspective view of the example intraoral positioning device in FIG. 1.

FIG. 3 depicts a side view of the example intraoral positioning device in FIG. 1.

FIG. 4 depicts a top view of the example intraoral positioning device in FIG. 1.

FIG. 5 depicts a front view of the example intraoral positioning device in FIG. 1.

FIG. 6 depicts a top view of the example intraoral positioning device in FIG. 1.

FIG. 7 depicts a front view of the example intraoral positioning device in FIG. 1.

FIG. 8 depicts a rear perspective view of the bottom section of another example intraoral positioning device.

FIG. 9 depicts a front view of the bottom section of the example intraoral positioning device in FIG. 8.

FIG. 10 depicts another view of the bottom section of the example intraoral positioning device in FIG. 8.

FIG. 11 depicts a rear perspective view of the upper section for the lower section of the example intraoral positioning device in FIG. 8.

FIG. 12 depicts a front view of the upper section for the lower section of the example intraoral positioning device in FIG. 8.

FIG. 13 depicts another view of the upper section for the lower section of the example intraoral positioning device in FIG. 8.

FIG. 14 depicts a front perspective view of another example intraoral positioning device.

FIG. 15 depicts a rear perspective view of the example intraoral positioning device in FIG. 14.

FIG. 16 depicts a top view of the example intraoral positioning device in FIG. 14.

FIG. 17 depicts a side view of the example intraoral positioning device in FIG. 14.

FIG. 18 depicts a front view of the example intraoral positioning device in FIG. 14.

FIG. 19 depicts a top view of the example intraoral positioning device in FIG. 14.

FIG. 20 depicts a rear view of the example intraoral positioning device in FIG. 14.

FIGS. 21-33 depict several views of an example adjustable intraoral positioning device (and individual components) for lateral tongue displacement.

FIGS. 34-46 depict several views of another example adjustable intraoral positioning device (and individual components) for vertical tongue displacement (e.g., tongue depression).

FIGS. 47-51 depict various views of an example intraoral positioning device that is customized for a patient.

FIGS. 52-56 depict various views of another example intraoral positioning device that is customized for a patient.

### DETAILED DESCRIPTION OF THE INVENTION

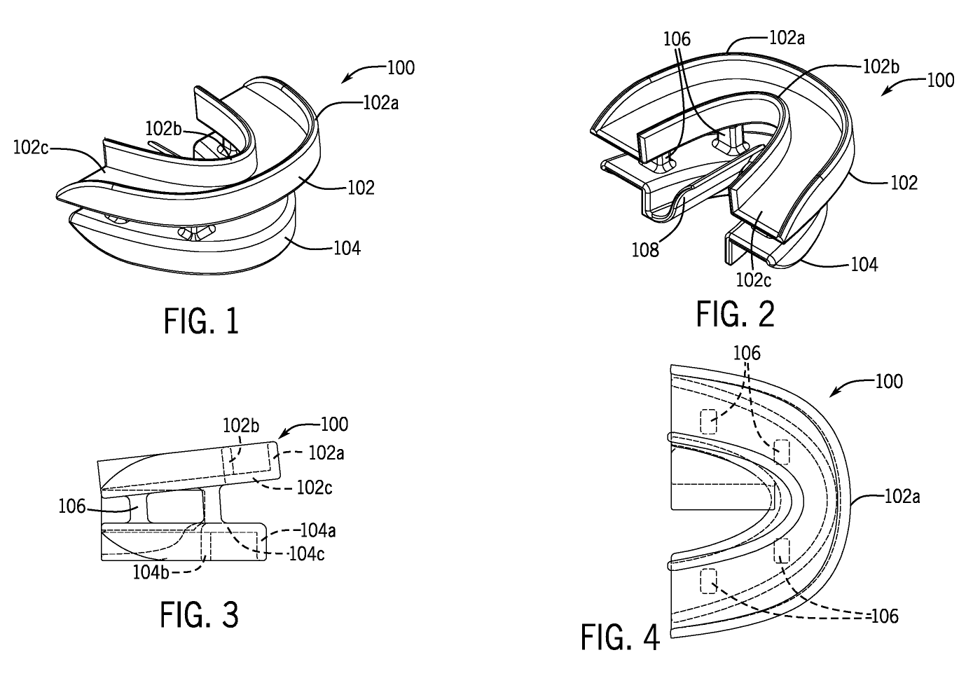

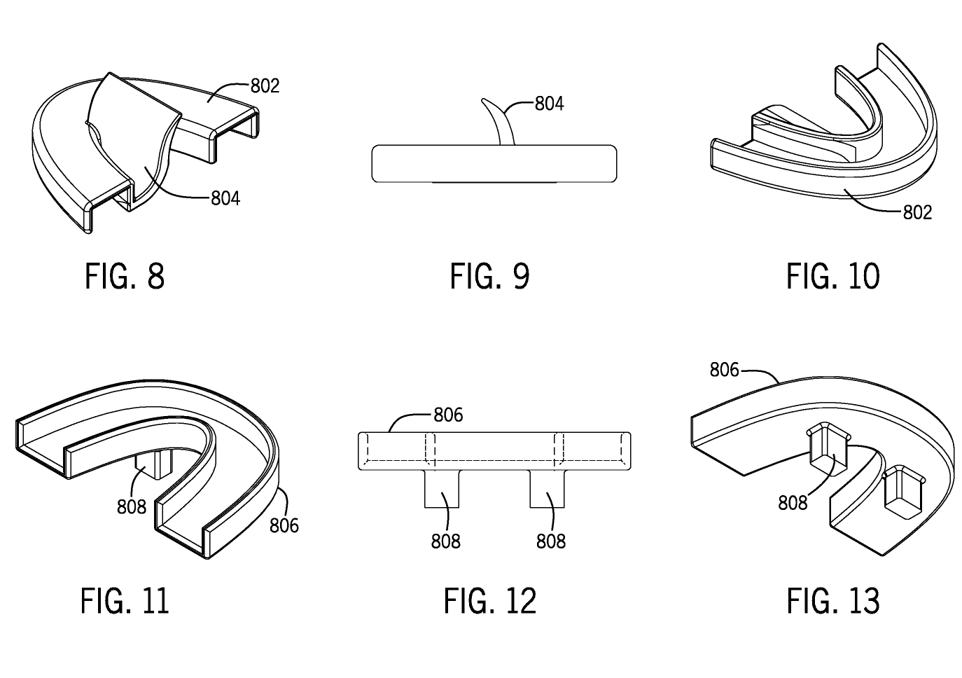

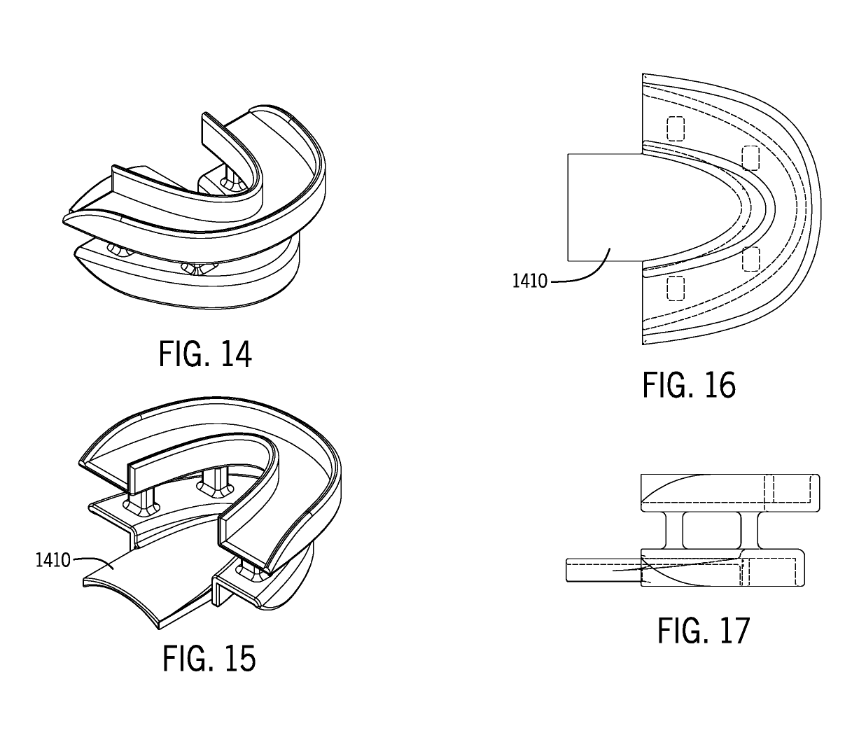

Embodiments of the present invention are described herein with reference to the drawing figures. FIGS. 1-7 depict various views of example intraoral positioning device (IPD) 100 that is used to position a patients jaw, mouth and soft tissue (e.g., tongue) for intensity-modulated radiation therapy (IMRT) planning and treatment. IPD 100 is a customizable device that reproducibly shields oral structures and displaces soft tissue shields (thereby sparing such tissue unnecessary exposure to radiation. IPD 100 is configured or manufactured as one-piece (integral), but those skilled in the art know that IPD 100 may be made in two or more pieces that are combined to form IPD 100.

IPD 100 comprises upper section 102 that is configured to position and shield upper oral structures such as teeth soft tissue (e.g., gums) and maxilla and a lower section 104 that is configured to position and shield the lower oral structures such as the teeth, soft tissue and a portion of the mandible. (Upper and lower sections are sometimes together referred to as a bite stent.) Upper and lower sections 102, 104 each have annular inner 102b, 104b and outer ledges 102a, 104a (walls), respectfully displaced by a generally annular flat support section 102c, 104c. Upper and lower sections 102,104 are separated by support posts 106. Support posts 106 are positioned to adequately separate upper and lower sections 102,104 to accommodate the natural position of a patient's jaws and provide adequate airflow for a user to breath. Upper section 102 extends beyond the length of lower section 104 to accommodate the natural position of the jaws as indicated above. As those skilled in the art know, the upper and lower sections 102, 104 may be parallel or the lower section 104 may extend further forward than upper section 102 to accommodate an under-bite (lower jaw extending beyond upper jaw) or vise-versa to accommodate an overbite.

In the example IPD 100, front posts 106 are configured to a length that is greater than rear support posts 106, whereby upper and lower sections 102,104 form an acute angle. In other words, upper and lower sections 102, 104 are not parallel to each other. However, those skilled in the art know that the support posts 106 may be configured at any length to configure upper and lower sections 102,104 to form any angle to accommodate the natural position of a patient's jaw (i.e., so as to contour to a particular oral structure of patient's mouth). Upper and lower sections 102, 104 may also be made parallel to other another.

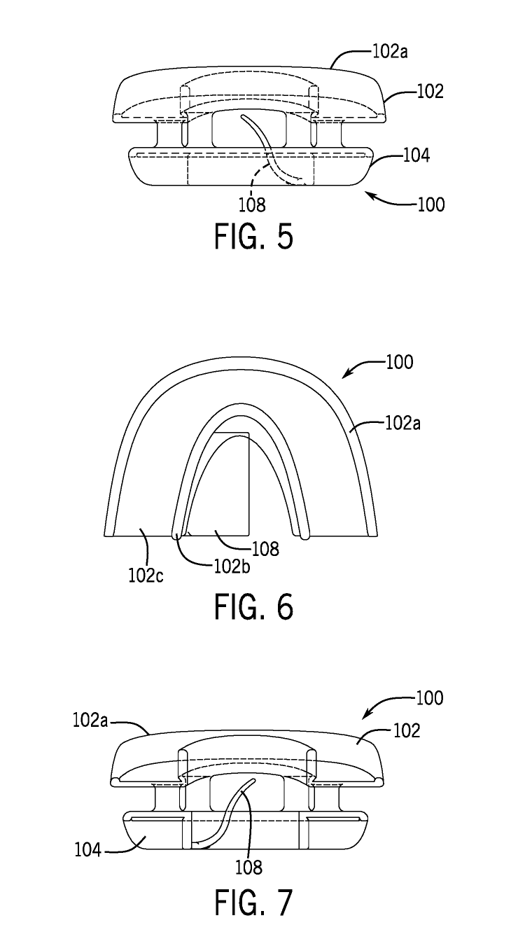

IPD 100 further comprises tongue displacement stent 108 to provide consistent displacement of oral soft tissue (e.g., tongue) that is not involved in IMRT planning and treatment. Stent 108 has an S-shape configuration whereby the lower edge thereof extends from the bottom edge of ledge 104b upwardly toward the center between upper and lower sections 102,104. Stent 108 is positioned along the lower right inside edge (facing front) but it may be configured along the lower left edge of ledge 104b. This is best viewed in FIGS. 5-7. The S-shape comprises a lower and upper curve, the lower cure having a concave shape to depress sublingual tissue and the upper curve having a convex shape to alleviate pressure point on the lingual tissue. The radii of these curves are adjusted as needed per patient as known to those skilled in the art.

FIGS. 8-13 depict various views of another example intraoral positioning device (IPD) 800 that is used to position a patients jaw, mouth and soft tissue (e.g., tongue) for intensity-modulated radiation therapy (IMRT) planning and treatment. IPD 800 is similar in shape and function as IPD 100, but IPD 800 is configured as separate upper and lower sections that are combined (e.g.,) together to form the complete device. FIGS. 8-10 depicts lower section 802 along with tongue displacement stent 804. FIGS. 11-13 depicts upper section 806 along with support posts 808. In this example, there are only two support posts but any number of posts may be used to achieve desired results. Posts 808 may have mating indents on lower section 802 for positioning or posts 808 may rest flat on lower section 802. In this example, the upper and lower sections 806, 802 are not secured to one another to facilitate insertion and removal with patients with special circumstances (e.g., smaller mouths, maxillofacial deformity, etc.).

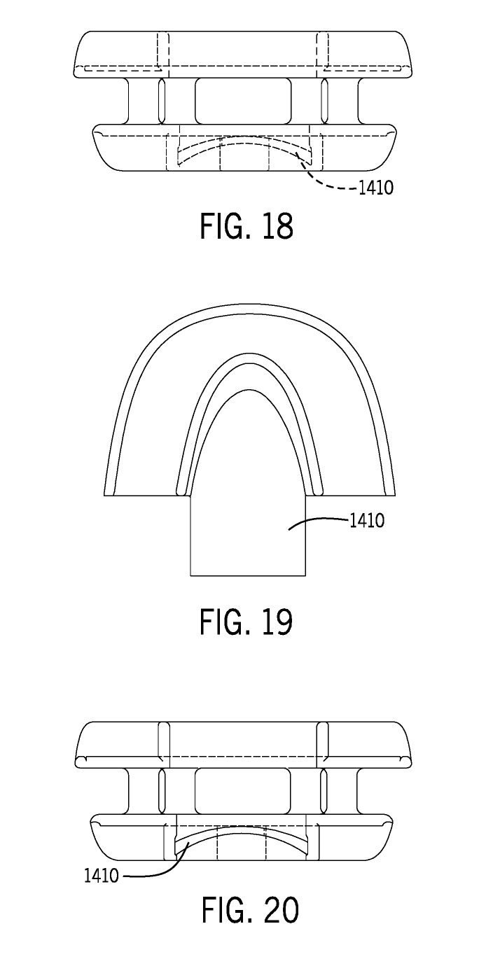

FIGS. 14-20 depict various views of another example intraoral positioning device (IPD) 1400 that is used to position a patients jaw, mouth and/or soft tissue (e.g., tongue or lingual tissue) for intensity-modulated radiation therapy (IMRT) planning and treatment. IPD 1400 is similar to IPD 100 in that the upper and lower sections are the same. Four support posts are similarly employed. However, upper and lower sections are configured parallel to one another. Alternatively, upper and lower sections may be angled as known to those skilled in the art. IPD 1400 further comprises tongue displacement stent 1410, but stent 1410 is a vertical depressing stent that extends across the bottom edge of the inner ledge of the lower section. Vertical stent 1410 has a convex curve to contour to the lingual tissue, thereby reducing the pressure points on such tissue. Stent 1410 is curved to fit a patient as needed. Alternatively, stent 1410 may be flat or concave as known to those skilled in the art.

The IPD devices (examples/embodiments) described herein may incorporate radiation-shielding properties to reduce radiation exposure to uninvolved tissue. For example, tungsten, bismuth or other materials known to those skilled in the art may be used for their radiation shielding properties.

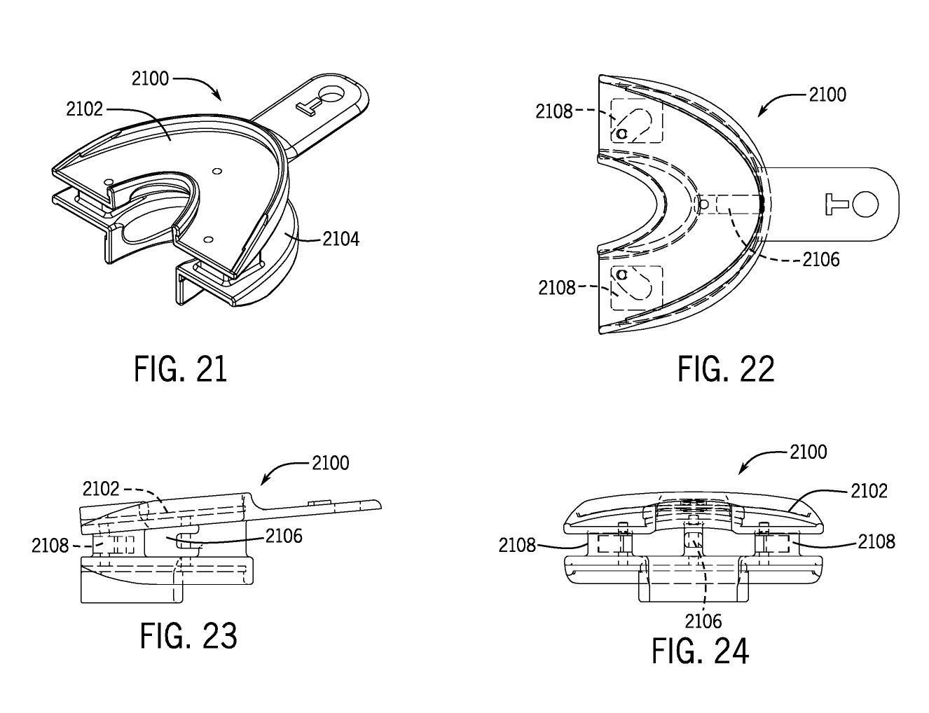

FIGS. 21-32 depict several views of example adjustable IPD 2100 (and components thereof). This IPD 2100 is configured so as to displace a patient's tongue laterally (IPD 2100 also referred to as lateral IPD). Similar to IPD 100 described above, IPD 2100 comprises upper section 2102 that is configured to position and shield upper oral structures such as teeth, soft tissue (e.g., gums) and maxilla and lower section 2104 that is configured to position and shield the lower oral structures such as the teeth, soft tissue and a portion of the mandible. Upper and lower sections each have annular inner ledges and outer ledges, respectfully displaced by a generally annular flat support section or wall for engaging the teeth bottom (as shown).

Upper and lower sections 2102, 2104 are separated by support posts 2106, 2108, similarly as described above. Support posts 2106, 2108 are positioned to adequately separate upper and lower sections 2102, 2104 to accommodate the natural position of a patient's jaws and provide adequate airflow for a user to breath. Upper section 2102 and lower section 2104 may be parallel or angled with respect to each other. Alternatively, lower section 2104 may extend further forward than upper section 2102 to accommodate an under-bite (lower jaw extending beyond upper jaw) or vice versa to accommodate an overbite.

Support post 2106 is specifically positioned mid-center between upper and lower sections 2102, 2104. Support post 2106 includes a lateral extension 2106-1 with a channel extending therethrough to receive a retaining pin as described below. Upper section 2102 and lower section 2104 each include first and second openings or holes. The first hole extends through the annular flat section of upper section and lower sections 2102, 2104 and they are positioned or configured to be aligned with the channel in post 2106 to receive retaining pin 2112 (to secure tongue displacement stent 2110 in place as described in detail below). The second hole in the upper and lower sections 2102, 2104 are aligned and positioned adjacent the edge or flat support walls for receiving retaining pin 2114 as described below. Support posts 2108 each have a curved indentation, recess or section to receive the adjustment member (2110-2) of a tongue displacement stent (2110) as it is adjusted for a patient (FIG. 27 best illustrates this). The adjustment member and tongue displacement stent are discussed in more detail below.

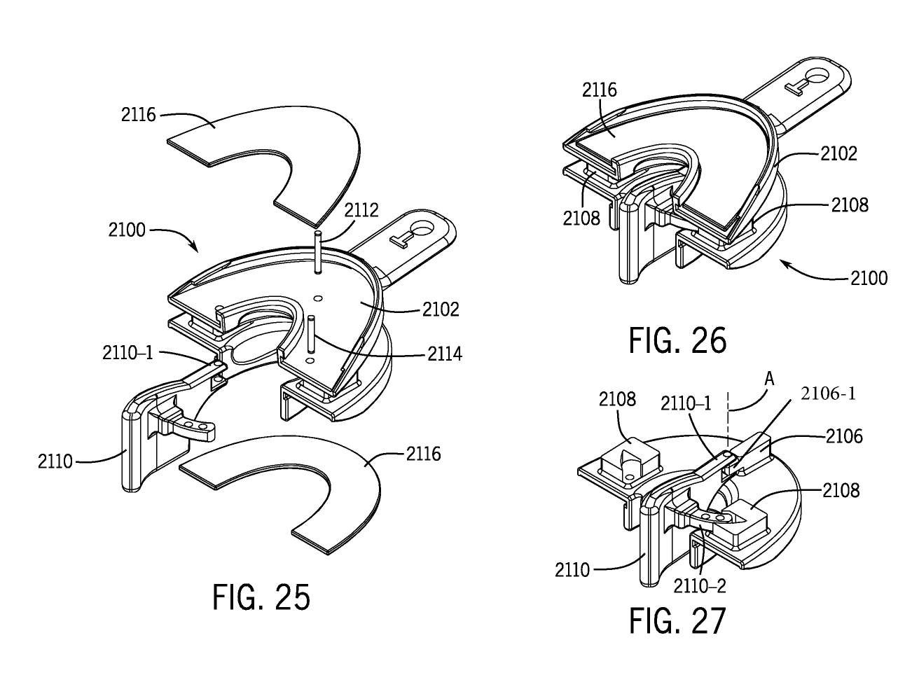

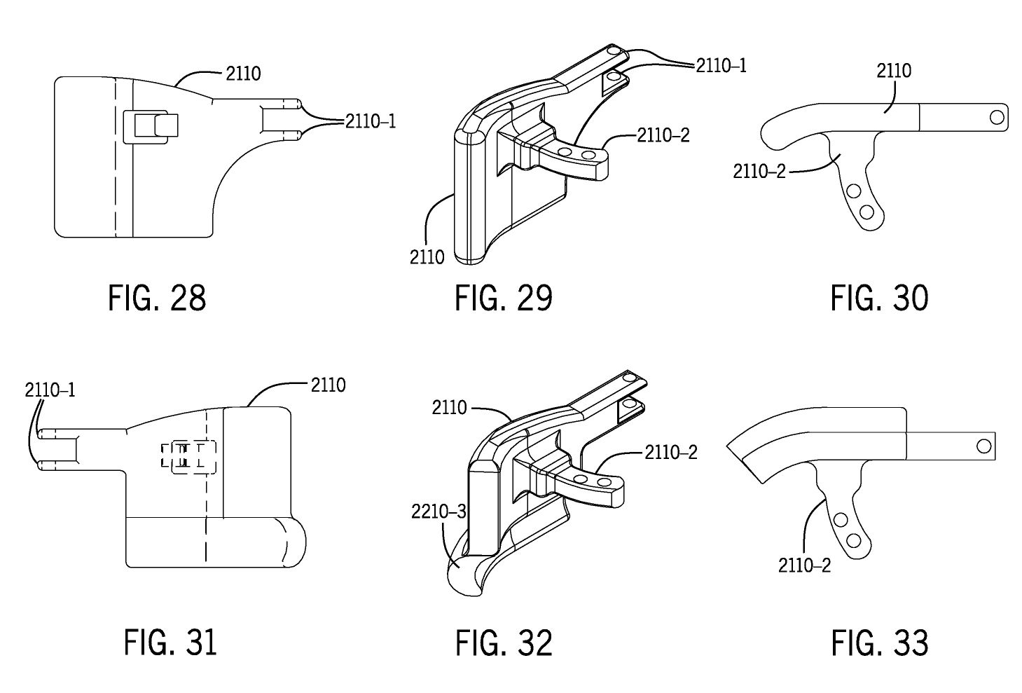

IPD 2100 further includes tongue displacement stent 2110 that may be adjusted to displace a patient's tongue laterally in either direction. (Note that FIGS. 21-24 do not show stent 2110 for illustration purposes.) Stent 2110 includes two extension arms 2110-1, each of which includes an opening or hole to engage IPD 2100 and to receive retaining pin 2112 that is inserted through the upper and/or lower sections 2102, 2104, thereby enabling tongue displacement stent 2110 to pivot about pivot point or axis A. This is best seen in FIG. 27 wherein the lower section 2104 is shown. Stent 2110 further includes adjustment member 2110-2 that has an arc shape that comprises a plurality of holes/channels there-along and extending there-through to receive retaining pin 2114 and secure stent 2110 in place and in a position as desired. FIGS. 25-27 depict an example tongue displacement stent 2110. (FIGS. 21-24 do now show the stent 2110.) FIGS. 28-30 and FIGS. 31-33 depict two other example stents (but same reference numerals 2110 etc.). These example stents are similar but the example in FIGS. 31-33 includes a curved lower ridge or raised portion 2210-3 to raise the tongue from the soft pallet. The curve may extend from front to rear to alleviate pressure points on the tongue.

IPD 2100 further includes retaining pins 2112, 2114 as identified above. Retaining pins 2110, 2112 is sized to through the holes in upper section 2102 and lower section 2104, but the diameter of retaining pin 2110, 2112 are slightly larger than the holes in upper and lower sections 2012, 2104 to maintain the position of the pins within such structures. Stent 2110 is configured to pivot about pivot axis A to a designated point and retaining pin 2114 is inserted into the channel through the opening in adjustment member 2110-2 to secure stent 2110 in place for use and therapy treatment.

A layer of thermo-formed material 2116 is shown (FIG. 25 for example) and it is positioned or fitted on both upper and lower sections 2102, 2104 of the IPD 2100 (covering flat across the upper and lower sections). IPD 2100 is then dipped in heated water between about 155-170 degrees Fahrenheit, or heated using another suitable method as known to those skilled in the arts and then placed in the patient's mouth to be formed.

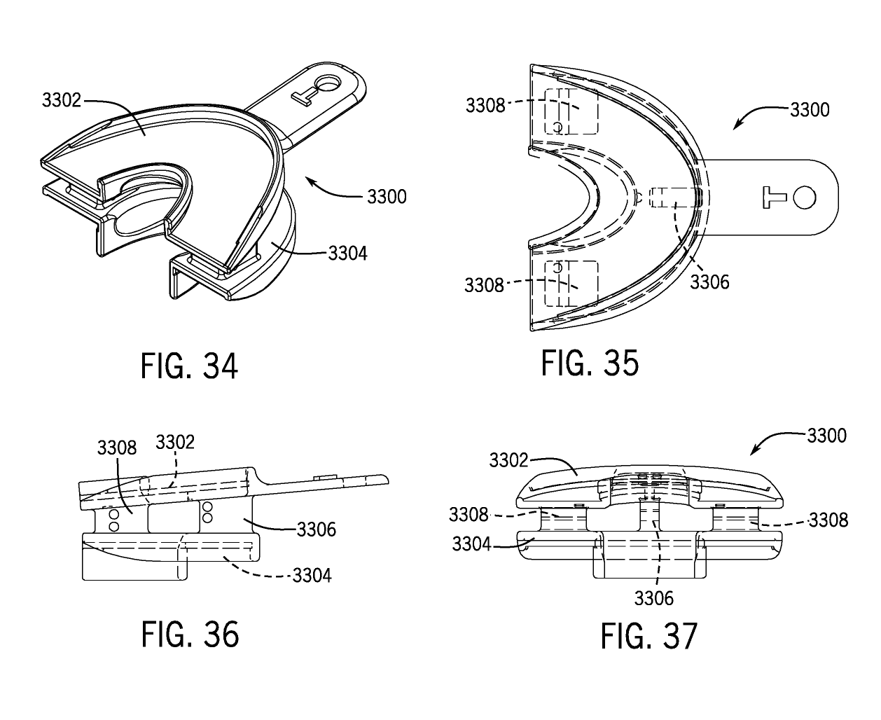

FIGS. 34-46 depict several views of another example IPD (and components). IPD 3300 is configured so as to displace a patient's tongue vertically (e.g., depressed into the base of the lower jaw, or lifted off the base of the lower jaw). (IPD 3300 is thus also referred to as vertical IPD). (Similarly, IPD 2100 as described above is configured so as to displace a patient's tongue laterally. IPD 2100 also referred to as lateral IPD). IPD 3300 comprises upper section 3302 that is configured to position and shield upper oral structures such as teeth, soft tissue (e.g., gums) and maxilla and lower section 3304 is configured to position and shield the lower oral structures such as the teeth, soft tissue and a portion of the mandible. Upper and lower sections each may have annular inner ledges and outer ledges, respectfully displaced by a generally annular flat support section or wall for engaging the teeth bottom similar to IPD 2100 above.

Upper and lower sections 3302, 3304 are separated by three support posts 3306, 3308 similarly as described above. Support posts 3306, 3308 are positioned to adequately separate upper and lower sections 3302, 3304 to accommodate the natural position of a patient's jaws and provide adequate airflow for a user to breath. Support post 3306 is positioned mid-center between upper and lower sections 3302, 3304. Post 3306 has two (or more) lateral channels that extend therethrough. The channels are configured to receive retaining pin 3310 to adjust the position of stent 3314. Posts 3308 each include two (or more) channels that extend therethrough. These channels are configured to receive retaining pin 3312 to secure stent 3314 in place as described in more detail below. Upper section 3302 and lower section 3304 are depicted in an angular position with respect to each other or may be parallel with respect to each other. Alternatively, lower section 3304 may extend further forward than upper section 3302 or reverse to accommodate an under-bite (lower jaw extending beyond upper jaw) or vise versa to accommodate an overbite.

IPD 3300 further includes tongue displacement stent 3314 that may be adjusted to displace a patient's tongue vertically in either direction. (Note that FIGS. 34-37 do not show stent 3314 for illustration purposes.) Stent 3314 includes two extension arms 3314-1, each of which includes openings or holes to engage post 3306 and to receive retaining pin 3310 that is inserted between the upper and/or lower sections 3302, 3304 through post 3306, thereby enabling tongue displacement stent 3314 to pivot. This is best seen in FIG. 40 wherein the lower section 3304 is shown. Stent 3314 further includes lateral member 3114-2 that is rectangular in shape and it comprises a lateral channel that extends through member 3114-2 to receive retaining pin 3312 and secure stent 3314 in place and in a position as desired. This is best seen in FIG. 36. The holes on the posts 3306, 3308 enable a user to adjust the position of stent 3314 to satisfy various patient tongue anatomies and various patient radiation treatment plans as known to those skilled in the art.

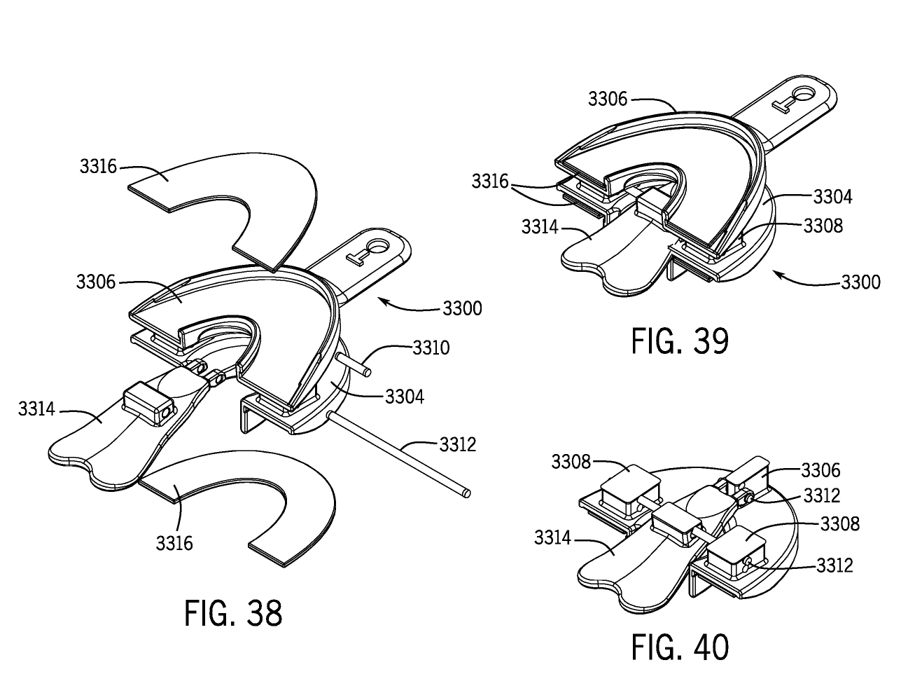

A layer of thermo-formed material 3316 is shown (FIG. 38 for example) and it is positioned or fitted on both upper and lower sections 3302, 3304 of the IPD 3300 (covering flat wall or upper and lower sections). IPD 3300 is then dipped in heated water between about 155-170 degrees Fahrenheit, or heated using another suitable method as known to those skilled in the arts, placed in the patient's mouth and formed.

Similar to the IPD 2100 above, a layer of thermo-formed material 3316 is shown (FIG. 25 for example) and it is positioned or fitted on both upper and lower sections 3302, 3304 of the IPD 3300 (covering flat wall or upper and lower sections). IPD 3300 is then dipped in heated water that between about 155-170 degrees Fahrenheit, or heated using another suitable method as known to those skilled in the arts, placed in the patient's mouth and formed.

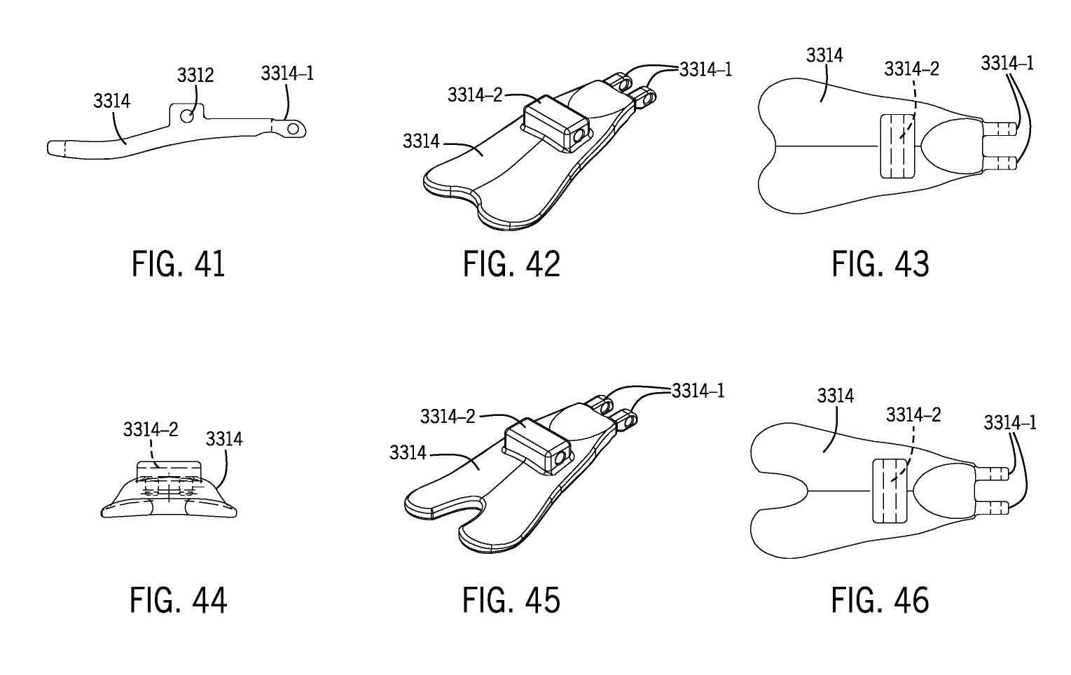

FIGS. 41-43 and 44-46 depict two examples of tongue displacement stents 3314 in detail.

In practice, a medical practitioner will select either the lateral or vertical IPD depending on whether a patient's tongue needs to be moved laterally to a side or elevated or depressed in accordance with the treatment plan and location of the tumor/designated area of treatment. The lateral IPD is curved with a lower raised portion to raise the tongue from the soft pallet. The curve may extend from front to rear to account for pressure points on the tongue. For example, a patient's tongue may need to be moved right laterally, with a slight separation from the lower soft pallet for a tumor that is in the left submandibular region (lower part of the jaw).

The vertical IPD may be configured as substantially flat configuration to provide even depressing force on the tongue. The vertical IPD may have a split design to accommodate the frenulum. The vertical IPD may have a flared back portion to disperse the force evenly across the tongue and may have a left right curve to contour to the natural shape of the tongue, with variations to fit specific applications.

Once a lateral or vertical IPD is selected, the IPD is pinned into place using the retaining pins. As described above, there are fixed adjustment points on each IPD that correspond to placement points in the adjustment member, which allows for fine adjustment and positioning. The positioning is then checked in the patient's mouth for fitment and comfort. A layer of thermo-formed material is placed on both upper and lower sections of the IPD on the flat walls thereof. The IPD is then dipped in heated water between about 155-170 degrees Fahrenheit, or heated using another suitable method as known to those skilled in the arts. The IPD is then placed into position in the patient's mouth. When the patient bites down the thermo-mold is set and the device is allowed to cool. The IPD is now ready for use.

Process of Diagnosis and Treatment. The process from initial diagnosis to treatment is now described for the IPDs as described herein. First, a physical mold or casting impression is made of patient's teeth or other body parts that need to be positioned and/or shielded. Alternatively, a digital scan or rendering (i.e., CT or MRI) scan is taken of the patient's mouth or body part. This information depicts or shows the structure of the body part that is to be positioned and/or shielded. Second, the digital or physical model of the structure that needs to be positioned and/or shielded is received along with the prescribed treatment plan from the doctor. Third, using a digital conversion process, a model or rendering of the IPD is created. Fourth, using 3D printing (sometimes with custom filaments) or casting techniques that incorporate proprietary radiation shielding element properties, the IPD is produced. At that point, the IPD is then available for testing and/or prescribed treatment. Adjustments may be made (filing or sanding are options to address any areas of discomfort).

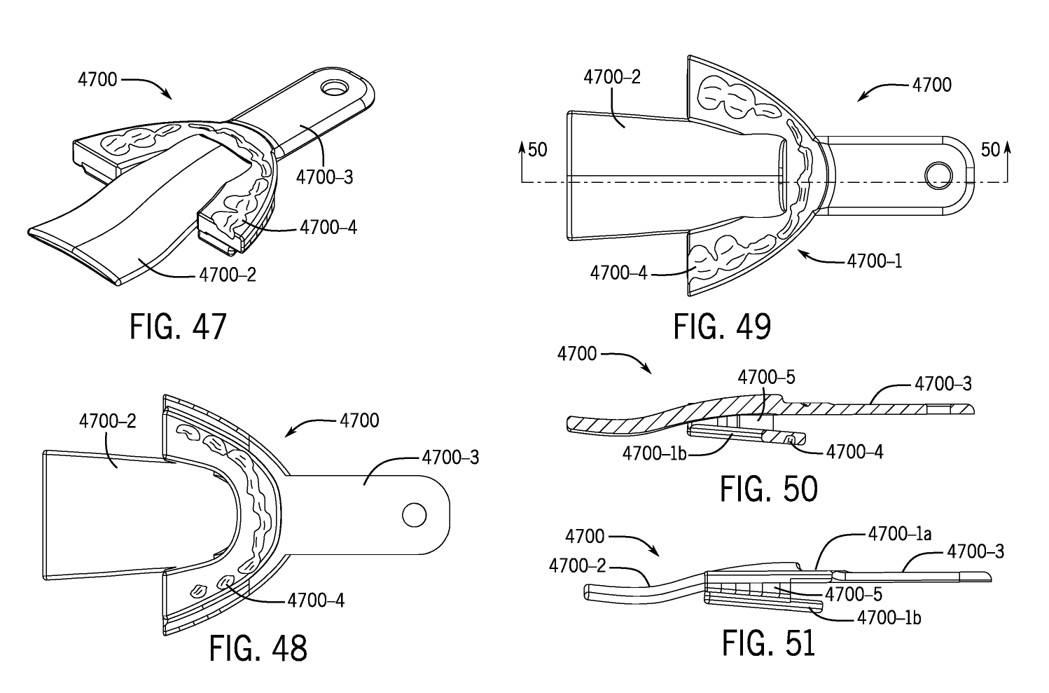

FIGS. 47-51 depict various views of an example customized IPD that is customized for a patient. In particular, using the method described below, the IPD is created as a customized device for a particular user (patient). In this respect, the IPD is designed and manufactured for a specific patient (user) based on the patient's anatomic structure and doctor's prescribed treatment plan. In this example, IPD 4700 is configured or manufactured as one-piece (integral) but may be created in multiple pieces or sections as known by those skilled in the arts.

In brief, the example of patient customized IPD 4700 is fitted within a patient's mouth to engage the patient's upper and lower jaws, mouth and soft tissue (e.g., tongue) for intensity-modulated radiation therapy (IMRT) planning and treatment. IPD 4700 is a device that reproducibly positions and shields oral structures by displacing soft tissue thus attenuating radiation exposure (thereby sparing such tissue unnecessary exposure to radiation). For description purposes, IDP 4700 is broken down into three sections or parts (components). These sections are (1) bite stent 4700-1 (section) having upper and lower sections or arches, (2) tongue displacement stent 4700-2 and (3) deployment handle or pull tab 4700-3.

FIG. 47 is a rear perspective view showing a final representation of IDP 4700 depicting the top set of teeth impressions 4700-4 that allow for precise positioning and a secure fit. Tongue displacement stent 4700-2 is a vertical depression stent with slight curve to position the tongue as prescribed. Specifically, tongue displacement stent 4700-2 has a shape that curves downwardly in convex manner to provide downward force upon the patient's tongue when stent 4700-2 is in place. This effectively immobilizes the tongue at the base of the mouth, at the same point during each IMRT procedure (i.e., over time, e.g., 30 treatment sessions over 6 weeks.) However, the curve of stent 4700-2 may have other shapes to achieve desired results as known those skilled in the art. For example, the stent may be angled with a steep drop toward rear of the mouth, it may be flat or it may curve upwardly toward the rear of the mouth for different anatomic structures and desired treatment positioning.

FIG. 48 depicts a bottom plan view of IPD 4700 illustrating a small size difference between the upper and lower arches (separated by separation blocks). IPD 4700 is part of the patient specific design that was adjusted based on the patient's anatomy, i.e., upper and lower jaw size. The lower set of teeth impressions 4700-4 is also portrayed. Physical or digital teeth dental impressions are taken which are then used or incorporated into the design of the stent prior to manufacture.

FIG. 49 depicts a top plan view of IPD 4700. FIG. 50 depicts a side cut away view of IPD 4700 in FIG. 49 along line 50-50 with upper and lower arches shown. IPD 4700 is shown extending to the rear (left) and the pull-tab 4700-3 extends to the front (right). Separation blocks 4700-5 are seen with gaps between them that allow for airflow, thereby allowing a patient or user to breath with the device in place. Teeth impressions 4700-4 are shown where the upper and lower teeth fit. FIG. 51 depicts a view of the angle where the patient's jaws are hinged.

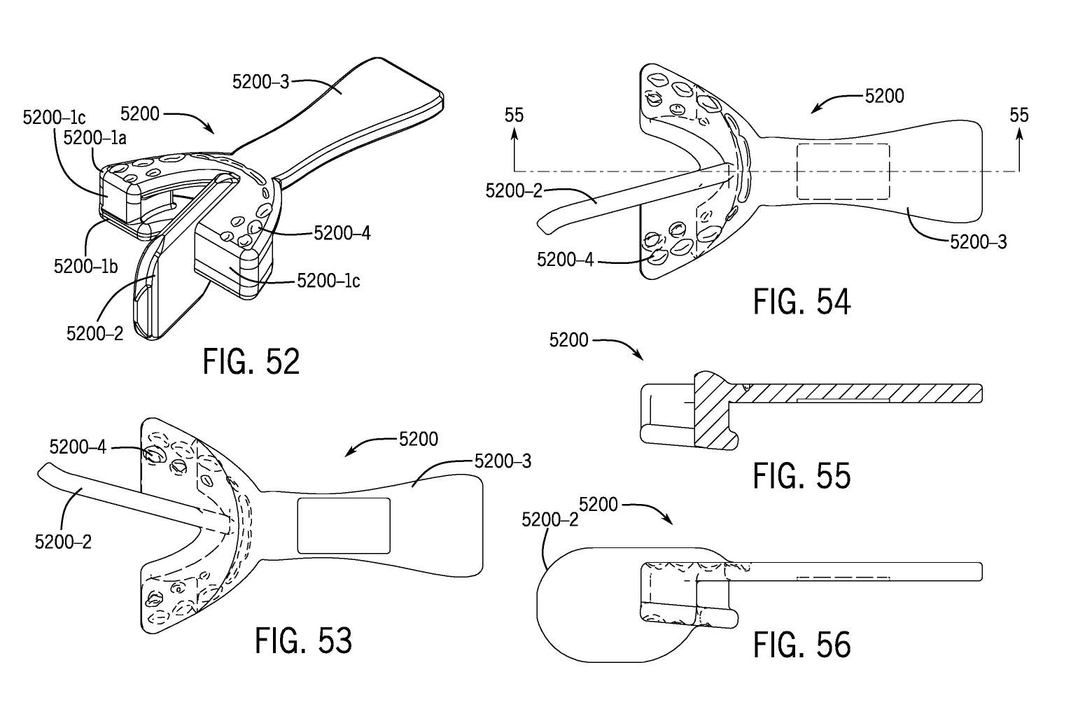

FIGS. 52-56 depict various views of another example intraoral positioning device that is customized for a patient. In particular, FIG. 52 depicts a rear perspective view of IDP 5200 illustrating upper and lower arches or sections 5200-1 and 5200-2. IDP 5200 is broken down into three sections or parts (component) similar to the embodiments above. These sections are (1) bite stent 5200-1 (section), (2) tongue displacement stent 5200-2 and (3) deployment handle or pull tab 5200-3. Bite stent 5200-1 (section) includes upper and lower sections or arches 5200-1a and 5200-1b and blocks or support posts 5200-1c that separate 5200-1a and 5200-1b. Support posts 5200-1c may also include holes that extend from the front to rear to accommodate saliva evacuation tubing that is placed through the device and positioned in the back of the patient's mouth allowing for accumulated saliva to be extracted with suction or other methods as known to those skilled in the art. Sections or arches 5200-1a and 5200-1b include teeth impressions 5200-4 that are configured to properly position upper oral structures such as teeth, soft tissue (e.g., gums) and maxilla and lower oral structures such as the teeth, soft tissue and a portion of the mandible, respectively.

Support posts 5200-1c are positioned to adequately separate upper and lower sections 5200-1a and 5200-1b to accommodate the natural position of a patient's jaws and provide adequate airflow for a user to breath. In the example shown, support posts 5200-1c are configured wills that are flush with upper and lower sections 5200-1a and 5200-1b to thereby reduce abrasion with a patient's soft tissue.

Upper section 5200-1a and lower section 5200-1b may be parallel or angled with respect to each other. Alternatively, lower section 5200-1b may extend further forward than upper section 5200-1a to accommodate an under-bite (lower jaw extending beyond upper jaw) or vice versa to accommodate an overbite.

Tongue displacement stent 5200-2 is a lateral (vertically) tongue displacement stent with a slight curve at the distal end to position the tongue as prescribed. Stent 5200-2 extends from apex of both the upper and lower sections (arches) and is connected to the support posts to create a singular continuous piece with no gaps or crevices, thus creating a flush union between parts. Stent 5200-2 alternatively may be curved, straight or rounded based on medical prescription or patient comfort.

FIG. 53 depicts a bottom plan view of IPD 5200 illustrating a small size difference between the upper and lower sections. IPD 5200 is part of the patient specific design that was adjusted based on the patient's anatomy, i.e., upper and lower jaw size. The lower set of teeth impressions 5200-4 is also portrayed. FIG. 54 depicts a top plan view and FIG. 55 depicts a side cut away view of IPD 520, respectively. IPD 5200 is shown extending to the rear (left) and pull-tab 5200-3 extends to the front (right). The separation posts 5200-1C are configured with a gap therebetween to allow for airflow, thereby allowing a patient or user to breath with the device in place. Teeth impressions 5200-4 are shown where the upper and lower teeth fit. The angle between upper and lower sections 5200-1 and 5200-2 is best depicted in FIG. 56. This is at a point where the patient's jaws are hinged. Thus, the inter-jaw relationship is unique to each patient and their anatomic structure. The side profile of lateral tongue displacement stent 5200-2 is shown extending left. Pull-tab 5200-3 extends to the right.

Importantly, the IPD 5200 and IPD 4700 are each a one piece system with configuration and smooth edges (lack of joints or small moving parts) that reduces crevices and small areas where debris can accumulate, thereby reducing abrasion of soft tissues which improve ease of use in an IMRT procedure. The one piece system may be manufactured as an integral component or several components bonded, glued or otherwise attached as known to those skilled in the art.

Also, the IPDs are described with respect to patient's having teeth. However, those skilled in the art know that the upper and lower sections of the embodiments described in this disclosure may use a channel or other structure to support an edentulous patient (without teeth). This might include soft molding putties or other support systems.

In addition, any of these IPD examples may be created with full upper and lower sections (arches) as described above or half sections (arches).

It is to be understood that the disclosure teaches examples of the illustrative embodiments and that many variations of the invention can easily be devised by those skilled in the art after reading this disclosure and that the scope of the present invention is to be determined by the claim(s) below.

1. An intraoral positioning device to be positioned within a patient's mouth for intensity-modulated radiation therapy (IMRT) planning and treatment, the intraoral positioning device comprising:
a bite stent having upper and lower sections, the upper section configured to engage an upper oral structure of the patient's mouth and the lower structure configured to engage an lower oral structure of the patient's mouth during IMRT planning and treatment;
a tongue displacement stent extending from and fixed to the bite stent, the tongue displacement stent configured to displace a tongue of the patient in a direction with respect to a base of the patient's mouth in accordance with a patient's prescription for IMRT planning and treatment, whereby the patient's tongue is positioned in a motionless state during the IMRT planning and treatment; and
a deployment handle extending from and fixed to the bite stent, the handle configured to be grasped to by a user so as to introduce and deploy the intraoral device into the patient's mouth for the IMRT planning and treatment and remove the device after IMRT planning and treatment.

2. The intraoral positioning device of claim 1 further comprising one or more support posts positioned between and configured to support the upper and lower sections.

3. The intraoral positioning device of claim 2 wherein the one or more support posts positioned between the first and second sections to support the upper and lower sections, the one or more posts having a block shape with one or more walls that are positioned to be flush with edge of the upper and lower sections.

4. The intraoral positioning device 1 wherein the tongue displacement stent is configured to displace the patients tongue in a horizontal direction.

5. The intraoral positioning device 1 wherein the tongue displacement stent is configured to displace the patients tongue in a vertical direction.

6. The intraoral positioning device of claim 1 wherein the upper and lower sections each define an arch shape to accommodate the upper oral structure and the lower oral structure of the patient's mouth, the upper and lower sections each define an apex in which an end of the tongue displacement stent is secured.

7. The intraoral positioning device of claim 7 wherein the tongue displacement stent extends outwardly from the apex.

8. The intraoral positioning device of claim 7 wherein the tongue displacement stent has a straight or curved shape.

9. The intraoral positioning device of claim 8 wherein the upper and lower sections each include teeth impression of the patient.

10. The intraoral positioning device of claim 9 wherein the teeth impressions are formed from a layer of thermo-formed material.

11. The intraoral positioning device of claim 1 wherein the upper and lower sections each define an arch shape to accommodate the upper oral structure and the lower oral structure of the patient's mouth and wherein the upper and lower sections each include an outer annular ledge and an inner annular ledge that are displaced by a flat support wall, wherein the tongue displacement stent extends from the lower section of the bite stent upwardly toward the upper section within an area of upper and lower sections defined by their arch shaped, whereby the displacement stent displacing the patient's tongue laterally.

12. The intraoral positioning device of claim 1 wherein the upper and lower sections each define an arch shape to accommodate the upper oral structure and the lower oral structure of the patient's mouth and wherein the upper and lower sections each include an outer annular ledge and an inner annular ledge that are displaced by a flat support wall, wherein the tongue displacement stent extends from the lower section of the bite stent upwardly toward the upper section within an area of upper and lower sections defined by their arch shaped, whereby the displacement stent displacing the patient's tongue vertically.

13. The intraoral positioning device of claim 11 wherein the tongue displacement stent has a straight, flat, or arc shape.

14. The intraoral positioning device of claim 2 wherein the one or more support posts include an opening therethrough for receiving tubing that enables saliva extraction.

15. An intraoral positioning device to be positioned within a patient's mouth for intensity-modulated radiation therapy (IMRT) planning and treatment, the intraoral positioning device comprising:
an upper section configured to engage an upper oral structure of the patient's mouth and a lower structure configured to engage a lower oral structure of the patient's mouth during IMRT planning and treatment;
a plurality of posts spaced between the upper and lower section, thereby supporting the upper and lower sections;
a tongue displacement stent configured to pivot with respect to the first and second sections so as to enable a user to adjust the position of the tongue displacement stent to a plurality of positions to displace the tongue of the patient in accordance with a patient's prescription for IMRT planning and treatment, whereby the patient's tongue is positioned in a motionless state during the IMRT planning and treatment; and
a deployment handle extending from and fixed the upper or lower sections, the handle configured to be grasped to by a user so as to introduce and deploy the intraoral device into the patient's mouth for the IMRT planning and treatment and remove the device after IMRT planning and treatment.

16. The intraoral positioning device of claim 15 wherein the upper and lower sections include a plurality of holes for receiving a plurality of retaining pins.

17. The intraoral positioning device of claim 15 wherein the plurality of posts includes a first post that is positioned mid-center between the upper and lower sections, the first post comprising a lateral extension with a channel that is aligned with a hole in the upper and lower sections to receive a retaining pin.

18. The intraoral positioning device of claim 17 wherein the tongue displacement stent comprises an the adjustment member having first and second extension arms, each of which having an opening that is aligned with the holes in upper and lower sections and channel in the first post so as to receive the retaining pin, thereby enabling the tongue displacement stent to pivot with respect to the upper and lower sections.

19. The intraoral positioning device of claim 17 wherein the adjustment member further includes a plurality of channels extending therethrough, each channel configured to receive a second retaining pin.

20. The intraoral positioning device of claim 19 wherein the plurality of posts includes a second post with an indentation for receiving the adjustment arm, thereby enabling a user to insert the second retaining pin through the openings in the upper and lower sections and through a channel within the adjustment arm to thereby secure the tongue displacement stent in place.

21. The intraoral positioning device of claim 13 wherein the plurality of posts includes a first post that is positioned mid-center between the upper and lower sections, the first post comprising a channel that is configured to receive a first retaining pin.

22. The intraoral positioning device of claim 21 wherein the tongue displacement stent includes first and second arms to engage with the first post, each arm has an opening to receive the first retaining pin, thereby enabling the tongue displacement stent to pivot vertically with respect to the upper and lower sections.

23. The intraoral positioning device of claim 22 wherein the plurality of posts includes second and third opposing posts that are positioned between the upper and lower sections, the second and third posts each comprise a plurality of channels configured to receive a second retaining pin to adjust the position of tongue displacement stent with respect to the upper and lower sections.

24. The intraoral positioning device of claim 23 wherein the tongue displacement stent further includes a member positioned midway on the stent, the member having a channel for receiving the second retaining pin that extends into the channels within the second and third posts.

25. The intraoral positioning device of claim 15 wherein the tongue displacement stent is configured to pivot laterally with respect to the first and second sections so as to enable a user to adjust the position of the displacement stent.

26. The intraoral positioning device of claim 15 wherein the tongue displacement stent is configured to pivot vertically with respect to the first and second sections so as to enable a user to adjust the position of the displacement stent.
